(19) United States
(12) Patent Application Publication
(10) Pub. No.: US 2015/0223970 A1
(43) Pub. Date: Aug. 13, 2015

(54) COLD ROLLER MASSAGE BALL

(71) Applicant: Pro Performance Sports, L.L.C., Carlsbad, CA (US)

(72) Inventor: Allen Keith Holland, Carlsbad, CA (US)

(21) Appl. No.: 14/459,486

(22) Filed: Aug. 14, 2014

Related U.S. Application Data
(60) Provisional application No. 61/939,553, filed on Feb. 13, 2014.

(51) Int. Cl. A61F 7/00; A61H 15/00

(52) U.S. Cl. CPC A61F 7/00; A61H 15/00; A61H 2015/0042; A61F 2007/0087

(57) ABSTRACT

A massager includes a ball captive in a cup adapted to be used with or without a holder. The ball contains a thermal liquid which may be refrigerated or frozen to provide a cold rolling ball massage. The holder and the cup may be thermally insulating to protect the user's hand from the cold temperature of the ball. The holder may have a bottom opening to allow the cup to be pushed out of the holder. Ridges on the holder may be used to provide an enhanced grip.

### PRIORITY CLAIM

This Application claims priority to U.S. Provisional Patent Application No. 61/939,553 filed Feb. 13, 2014 and incorporated herein by reference.

### BACKGROUND OF THE INVENTION

Massage is used to relieve muscle and tendon inflammation. Massage may improve blood circulation in muscles which can assist healing. Massage is also used for so-called trigger point conditions characterized by a knotted muscle or a tight muscle will not relax. Trigger points may inflame nerves and cause significant pain. Various massage balls, rollers and other devices have been used to a provide massaging actions not easily provided by hand. While these devices have met with differing degrees of success, improvements in massage devices are needed.

### SUMMARY OF THE INVENTION

A massager includes a ball captive in a cup adapted to be used with or without a holder. The ball contains a thermal liquid which may be refrigerated or frozen to provide a cold rolling ball massage. The holder and the cup may be thermally insulating to protect the user's hand from the cold temperature of the ball. The holder may have a bottom opening to allow the cup to be pushed out of the holder. Ridges on the holder may be used to provide an enhanced grip.

### BRIEF DESCRIPTION OF THE DRAWINGS

In the drawings, the same reference number indicates the same element in each of the views.

FIG. 1 is a top and side perspective view of a massager.

FIG. 2 is a top and side exploded perspective view of the massager of FIG. 1.

FIG. 3 is a side section view of the massager of FIG. 1.

FIG. 4 is a top perspective section view of the massager of FIG. 1.

FIG. 5 is a bottom perspective view of the massager of FIG. 1.

FIG. 6 is a top perspective view of the cup shown in FIGS. 1-5.

FIG. 7 is a top perspective view of the holder shown in FIGS. 1-5.

FIG. 8 is a top and side perspective view of a massager having an alternative ball design.

FIG. 9 is a top perspective view of the cup and ball shown in FIG. 8.

FIG. 10 is a side section view of the massager of FIG. 8.

FIG. 11 is an exploded perspective view of the ball shown in FIGS. 8-10.

FIG. 12 is a top and side perspective view of the massager of FIG. 8 with the holder removed from the cup.

FIG. 13 is a perspective view of the ball shown in FIGS. 8-13.

### DETAILED DESCRIPTION OF THE DRAWINGS

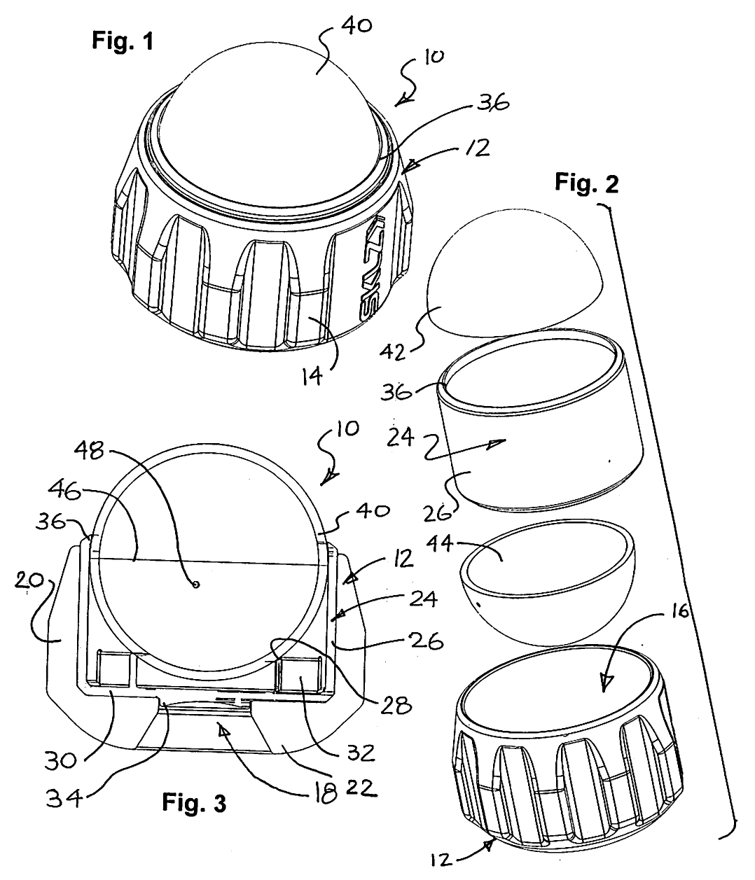

As shown in FIGS. 1-3, a massager 10 includes a ball 40 in a cup 24, with the cup insertable into and removable from a holder 12. The holder 12 has sidewalls 20 forming a cup opening 16, with the holder sidewalls 20 joined to or transitioning into a holder bottom 22. The cup opening 16 may be cylindrical, having circular sidewalls and a flat bottom. A central through hole or opening 18 may be provided in the holder bottom 22. The holder bottom may have a flat annular surface around the opening 18 so that the holder 12 sits fixed and upright on a flat surface. The opening may taper conically inwardly from the holder bottom 22 into the cup opening 16. Referring also to FIG. 7, a pattern of ridges 14 and grooves, or other gripping surfaces may be provided around the outside of the holder sidewalls 20. The holder 12 is typically rubber or plastic, or another resilient or flexible and thermally insulating material.

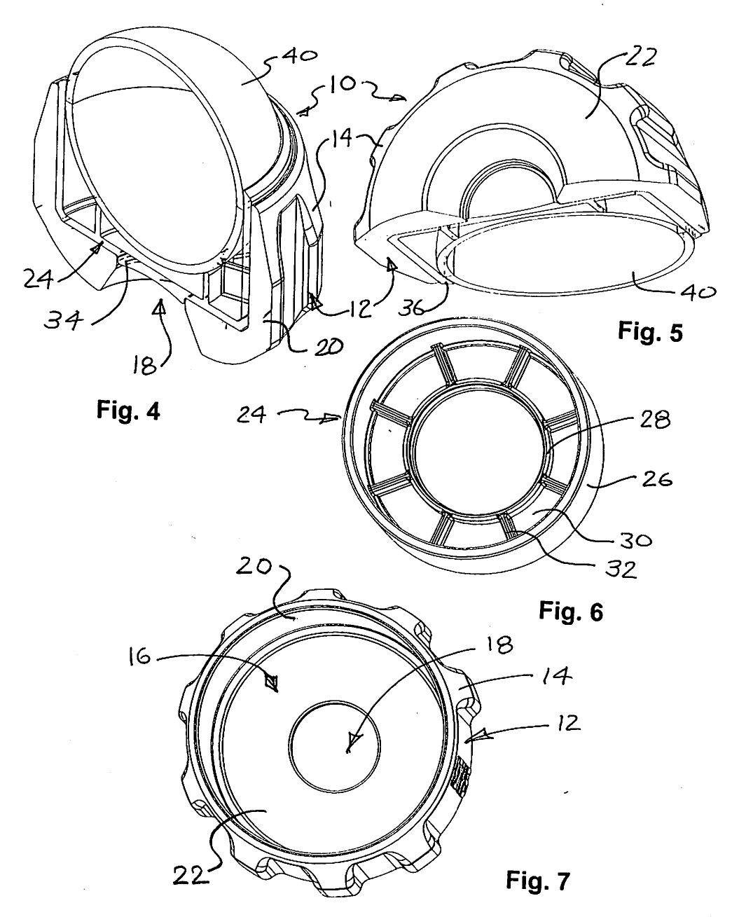

Turning to FIGS. 2-6, the cup 24 may have sidewalls complementary to the cup opening 16, so that the cup 24 may be moved into and out of the holder 12. In the example shown in FIG. 6, the cup 24 has cylindrical sidewalls 26 joined to a flat cup floor 30. An annular ball seat 28 is centrally located on the cup floor 30. Gussets 32 may be joined to the ball seat 28 and to the cup sidewalls 26. As shown in FIGS. 3 and 4, a cup hub 34 may project out from the back or bottom side of the cup floor 30, with the hub 34 extending slightly into the bottom opening 18 of the holder 12.

As shown in FIGS. 3 and 5, a cup lip 36 is provided at the top of the cup sidewalls 26. The diameter of the cup lip 36 is less than the diameter of the ball 40. The cup 24 may or may not be a thermally insulating material. The cup 24 is made sufficiently rigid so that the ball 40 can roll on the ball seat 28 in ordinary use. The ball 40 may be pressed into the cup 24 with the cup lip 36 momentarily flexing outwardly, and then returning to its original diameter, to retain the ball within the cup 24.

As shown in FIG. 3, with the cup 24 in the holder 12, the hub 34, if used, is inserted into the bottom opening 18 of the holder 12, and the cup floor 30 is flat against the inside of the holder bottom 22. Also as shown in FIG. 3, the top end of the cup sidewalls 26 projects slightly above the top end of the holder sidewalls 20, by about 2-10 mm. The ball 40 is rotatably supported from below on the ball seat 28. The ball is rotatably supported on the sides by the upper inner surfaces of the cup sidewalls 26 and/or the cup lip 36. The center line or equator 46 of the ball 40 is below the cup lip 36 and may also be below the top end of the holder sidewalls 20. Typically, with a ball 40 of radius R, the ball projects above the cup lip 36 by 0.6-0.9 R. The holder 12 is advantageously sufficiently flexible to allow the cup to fit securely within the holder 12 over the range of temperatures encountered in ordinary use.

The ball 40 contains a thermal liquid, i.e., a liquid having a thermal capacity equal to or greater than water. The thermal liquid may be water. The thermal liquid may also be a mixture of water and propylene glycol. The ball 40 may be manufactured using various techniques, with the specific technique used not material to the invention. In the example shown, the ball 40 is formed by two hemispheres joined together. These may be steel or other metal joined via friction or other form of welding or adhesives.

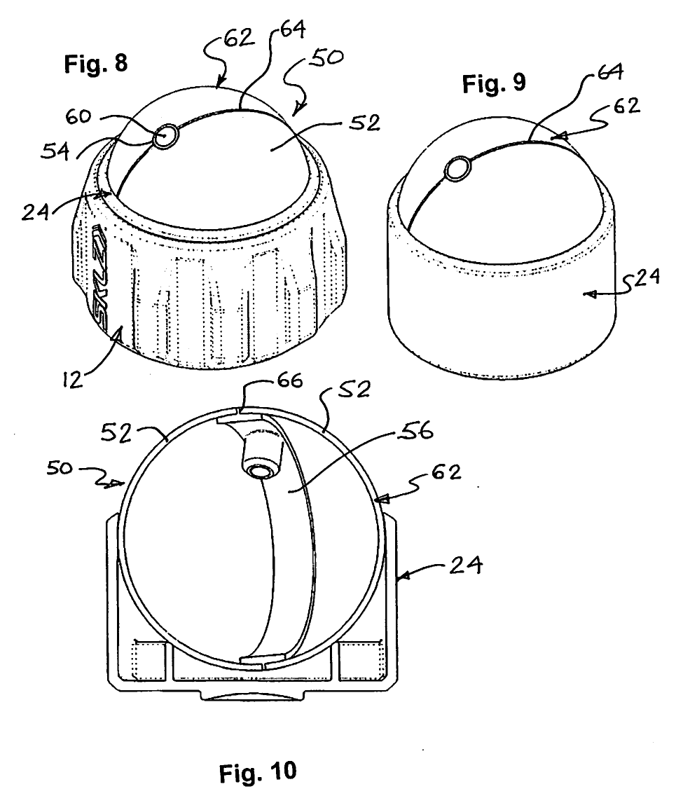

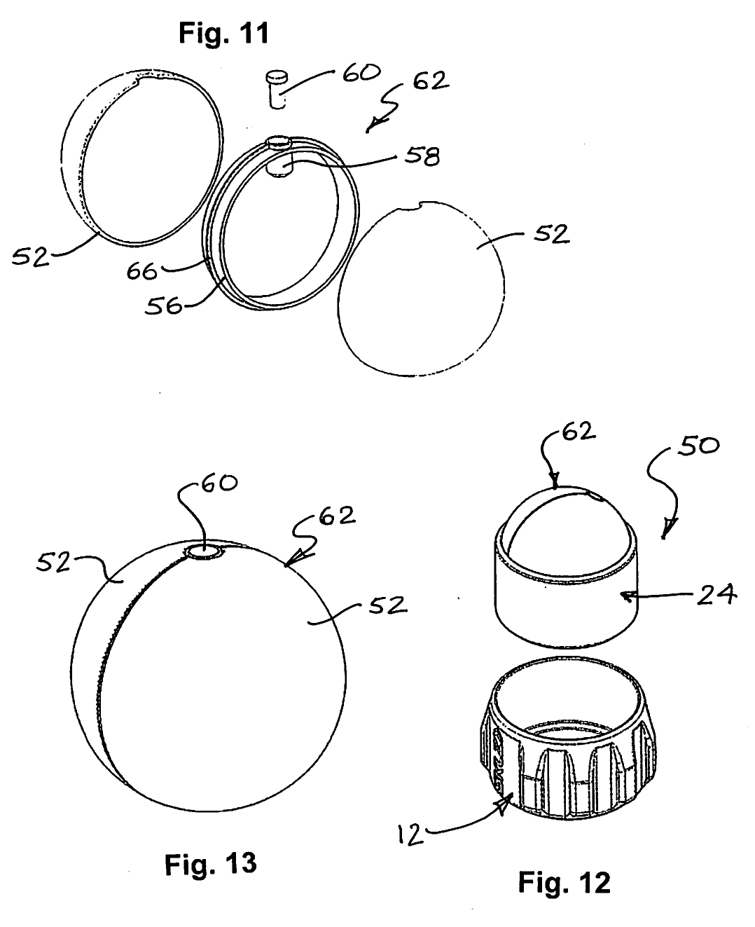

As shown in FIGS. 8-13, an alternative massager 50 may have a ball 62 formed via left and right hemispherical shells 52 joined to an internal ring 56. The internal ring 56 may have a ring flange 66, so that the internal ring has an inverted T-shaped cross section, as shown in FIG. 10. Referring to FIGS. 10 and 11, a fill tube 58 may extend through the internal ring 56, with each shell having cutout 54 to accommodate the fill tube. The shells 52 may be joined to the ring 56 via adhesives, welding, brazing, or other techniques. After the ball 62 is formed, the thermal liquid is introduced into the ball through the fill tube 58. The fill tube 58 may then be closed or capped via a plug 60 permanently attached to and sealing with the fill tube 58. The plug 60 may be pressed into the fill tube 58, optionally in combination with use of adhesives, welding, etc. The ball 62 may have a visible seam line 64.

The hemispheres may also be joined using an internal seam ring. In this case, a fill fitting or one way valve may optionally be provided on the seam ring, and both hemispheres may be joined to the seam ring.

The method used for filling the ball 40 with a thermal liquid may also vary. In the design shown a fill hole 48 is made in one of the hemispheres. After the hemispheres are joined to form the ball 40, the thermal liquid is introduced into the ball via the fill hole 48. The fill hole 48 may then be permanently plugged or closed, so that the thermal liquid is permanently sealed inside of the ball 40. The ball is generally made of a material having high thermal conductivity, such as a metal. The ball 40 may be partially or entirely filled with the thermal liquid.

In use, the cup 24 containing the ball 40 is placed into a freezer or refrigerator to chill or freeze the thermal liquid. The cup 24 is then removed from the freezer and inserted into the holder 12 as shown in FIG. 1. With the user's hand holding the holder 12, the user provides a cold rolling massage. The user's hand is insulated from the cold cup 24 and ball 40 by the holder 12. The cold temperature of the ball 40 may improve the massaging effect. As the massager 10 is moved over the skin surface, the ball 40 rolls within the cup 24. This avoids rubbing and also keeps the surface of the ball contacting the skin cold. After the massage is complete the cup 24 containing the ball 40 may be removed from the holder 12 by pushing the cup 24 out of the holder 12 via a finger or thumb inserted through the bottom opening 18. The cup with the ball may then be again put into a refrigerator or freezer.

In a modified design, the cup 24 may be made with sufficiently low thermal conductivity so that it can be comfortably handled from a freezer or refrigerator, without using the holder 12. In this design the holder 12 may be omitted or not used.

Thus novel massagers and methods have been shown and described. Various changes and substitutions may be made without departing from the spirit and scope of the invention. The invention, therefore, should not be limited, except to the following claims and their equivalents.

1. A massager comprising:
a holder;
a cup in a cup opening in the holder;
a ball rotatably supported by and retained in the cup, with the ball containing a thermal liquid.

2. The massager of claim 1 with the holder having a bottom opening to allow the cup to be pushed out of the cup opening, to remove the cup retaining the ball from the holder.

3. The massager of claim 1 with the holder and the cup comprising a thermal insulating material and the ball comprising a metal.

4. The massager of claim 3 wherein the ball is hollow and is substantially entirely filled with the thermal liquid.

5. The massager of claim 1 wherein the cup opening is cylindrical and further including ridges on an outer surface of the holder, with the ridges substantially parallel to a central axis of the cup opening.

6. The massager of claim 1 with the ball supported on an annular ball seat in the cup, and further including a plurality of radial gussets each having an inner end joined to the ball seat and an outer end joined to a cylindrical wall of the cup.

7. The massager of claim 1 with the ball retained in the cup via cup lip having a diameter less than the diameter of the ball.

8. The massager of claim 7 with the ball having a diameter of 2.5 to 15 cm.

9. The massager of claim 2 with the holder having a flat bottom surface and with the bottom opening having a conical taper centered on the flat bottom surface.

10. The massager of claim 2 further including a hub on a flat bottom surface of the cup, with the hub inserted into the bottom opening.

11. The massager of claim 1 with the ball comprising two hemispheres joined together at a seam line and a plugged fill hole on one of the hemispheres.

12. The massager of claim 1 with the cup adapted to be pressed into the holder and removed from the holder by hand.

13. The massager of claim 11 with the thermal liquid comprising water and propylene glycol.

14. The massager of claim 12 with the ball having a smooth spherical surface.

15. A massager comprising:
a holder having a flat bottom joined to cylindrical sidewalls forming a cylindrical cup opening, with the holder having a plurality of spaced apart ridges on an outer surface of the cylindrical sidewalls, and a center hole through the flat bottom;
a cup slidable into and out of the cup opening in the holder, with the cup having a ball seat on a cup bottom wall and cylindrical cup sidewalls, with a bottom end of the cylindrical cup sidewalls joined to the cup bottom wall, and with a cup lip at a top end of the cylindrical cup sidewalls;
a ball rotatably supported on the ball seat in the cup, and the ball retained in the cup via cup lip, wherein the ball is hollow and is substantially entirely filled with a thermal liquid, and the ball has a diameter greater than the cup lip.

16. The massager of claim 15 with the holder comprising rubber or plastic, the cup comprising plastic, and the ball comprising a metal.

17. The massager of claim 15 further including a plurality of radial gussets in the cup with each gusset having an inner end joined to the ball seat and an outer end joined to the cylindrical cup sidewall.

18. The massager of claim 15 with the thermal liquid comprising water and propylene glycol.

19. The massager of claim 15 with the ball comprising metal and having a smooth spherical surface.

20. The massager of claim 15 further including a hub on the cup bottom wall inserted into the center hole.
